SEQUENCE LISTING (12 sequences)

SEQ ID NO: 1 - DNA, 19 bp, Artificial Sequence
  Synthetic primer
gcagcaatcatgtgtccca

SEQ ID NO: 2 - DNA, 19 bp, Artificial Sequence
  Synthetic primer
tagcatcgcagacaaggtc

SEQ ID NO: 3 - DNA, 18 bp, Artificial Sequence
  Synthetic primer
agttgttcatcatcgagc

SEQ ID NO: 4 - DNA, 23 bp, Artificial Sequence
  Synthetic primer
catttccaagaaacaccttaggg

SEQ ID NO: 5 - DNA, 28 bp, Artificial Sequence
  Synthetic primer
caggacacaaagcttgtattacagactt

SEQ ID NO: 6 - DNA, 23 bp, Artificial Sequence
  Synthetic primer
catttccaagaaacaccttaggg

SEQ ID NO: 7 - DNA, 19 bp, Artificial Sequence
  Synthetic primer
gagatcatccaagaatcgc

SEQ ID NO: 8 - DNA, 18 bp, Artificial Sequence
  Synthetic primer
ggtagctgaattctgctg

SEQ ID NO: 9 - DNA, 18 bp, Artificial Sequence
  Synthetic primer
gagttgtttctcgagcag

SEQ ID NO: 10 - DNA, 18 bp, Artificial Sequence
  Synthetic primer
tgatcttttgcttgcagg

SEQ ID NO: 11 - DNA, 20 bp, Artificial Sequence
  Synthetic primer
tcgtggaggaaacrrcagrg

SEQ ID NO: 12 - DNA, 20 bp, Artificial Sequence
  Synthetic primer
tgtagcttccaccacctcca

(19) United States
(12) Patent Application Publication
(10) Pub. No.: US 2006/0069045 A1
(43) Pub. Date: Mar. 30, 2006

(54) Methods for treating familial dysautonomia

(76) Inventors: Berish Y. Rubin, Monsey, NY (US); Sylvia L. Anderson, Cresskill, NJ (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/221,130

(22) Filed: Sep. 7, 2005

Related U.S. Application Data
(60) Provisional application No. 60/609,151, filed on Sep. 10, 2004.

(51) Int. Cl. A61K 31/7048; A61K 31/353; A61K 31/355

(52) U.S. Cl. 514/27; 514/456; 514/458

(57) ABSTRACT

The present invention provides methods for modulating mRNA splicing in a normal or diseased cell or individual, e.g., elevating wild-type IKBKAP transcripts and the level of functional IKAP protein in an individual suffering from Familial Dysautonomia, by providing catechins, such as EGCG, to the cell or individual. The present invention also provides methods for treating Familial Dysautonomia by providing EGCG-related catechins to an individual having Familial Dysautonomia. Related therapeutic kits are also provided.

### CROSS REFERENCE TO RELATED APPLICATION

This application claims priority from U.S. Provisional Application No. 60/609,151, filed on Sep. 10, 2004.

### FIELD OF THE INVENTION

The present invention relates to the use of catechins for modulating mRNA splicing, particularly for elevating the level of wild-type IKBKAP-encoded transcript and functional IKAP protein, which is beneficial to individuals suffering from Familial Dysautonomia (FD). The present invention also relates to methods and kits for treating Familial Dysautonomia.

### BACKGROUND OF THE INVENTION

Familial dysautonomia (FD) is an autosomal recessive disorder primarily confined to individuals of Ashkenazi Jewish descent that affects the development and survival of sensory, sympathetic and some parasympathetic neurons (Riley et al., Pediatrics 3:468-77 (1949); Axelrod et al., Adv. Pediatr. 21:75-96 (1974); Axelrod, Familial Dysautonomia in: D. Roberston et al. (Eds.), Primer on the Autonomic Nervous System, Academic Press, San Diego, 1996, pp. 242-49). FD is caused by mutations in the gene termed IKBKAP which encodes a protein termed IKB kinase complex-associated protein (IKAP) (Anderson et al., Am. J. Hum. Genet. 68:753-58 (2001); Slaugenhaupt et al., Am. J. Hum. Genet. 68:598-605 (2001)). IKAP, which was originally reported to be a scaffold protein involved in the assembly of the Icb kinase complex (Cohen et al., Nature 395:292-97 (1998)), is more likely a component of the Elongator complex (Otero et al., Mol. Cell 3:109-18(1999); Hawkes et al., J. Biol. Chem. 277:3047-52 (2002)) and/or is a c-Jun N-terminal kinase (JNK)-associated protein (Holmber et al., J. Biol. Chem. 277:31918-28 (2002)).

Mutations that affect RNA splicing are a major cause of human genetic diseases. While many of these mutations result in what appears to be an absolute absence of the appropriately spliced gene product, in some cases mutations that affect splicing result in a milder form, or an adult onset form, of the disease in which “leaky” alternative mRNA splicing is observed that produces both mutant (skipped exon) and wild type (full length) transcripts (Huie et al., Biochem. Biophys. Res. Commun. 244:921-27 (1998); Boerkoel et al., Am. J. Hum. Genet. 56:887-97 (1995); Beck et al., Hum. Mutat. 14: 133-44 (1999); Kure et al., J. Pediatr. 137:253-56 (2000); Svenson, et al., Am. J. Hum. Genet. 69:1407-09 (2001); Svenson, et al., Am. J. Hum. Genet. 68:1077-85 (2001)).

FD is caused by one of the two known mutations. The most prevalent causative, or major FD-causing mutation, termed 2507+6T→C or IVS20^{+6T→C}, changes the sequence of the splice donor element of intron 20 from the consensus GTAAGT to a non-consensus GTAAGC, resulting in aberrant splicing generating a transcript lacking exon 20 and as a result a truncated protein. This mutation appears to be somewhat leaky as both the mutant and wild-type transcripts are detected in lymphoblasts of individuals homozygous for this FD-causing mutation (Slaugenhaupt, et al., 2001). The less common or minor mutation is a G→C transversion that results in an arginine to proline substitution of amino acid residue 696 of IKAP.

Regulated alternative splicing of pre-mRNA is a critical mechanism by which functionally different proteins are generated from the same gene. Pre-mRNA splicing is carried out by spliceosomes which are multi-component ribonucleoprotein (RNP) complexes containing small nuclear RNAs and a large number of associated proteins. Splice site selection and specificity are influenced by 5′ and 3′ splice sites located at the exon-intron boundaries of pre-mRNAs and by exonic splicing enhancer (ESE) and suppressor (ESS) elements (Blencowe, Trends Biochem. Sci. 25:106-10 (2000); Reed, Curr. Opin. Cell Biol. 12:340-45 (2000); Will and Luhrmann, Curr. Opin. Cell. Biol. 13:290-301 (2001); Maniatis and Tasic, Nature 418:236-43 (2002)).

In general, the binding of serine/arginine rich proteins (SR proteins) to the ESEs enhances splicing and the binding to the ESSs by members of the heterogeneous nuclear ribonucleoprotein (hnRNP) family results in a suppression of splicing. In vitro and in vivo studies reveal that SR proteins stimulate the selection of intron-proximal 5′ splice sites in pre-mRNAs that contain two or more alternative 5′ splice sites, while hnRNPs have the opposite effect, promoting the selection of intron-distal 5′ splice sites (Mayeda and Krainer, Cell 68: 365-75 (1992); Caceres et al., Science 265: 1706-09 (1994); Yang et al., Proc. Natl. Acad. Sci. USA 91: 6924-28 (1994)). The extensively studied hnRNPs of the A/B group exhibit significant amino acid sequence homology and changes in cellular amounts or activities of these proteins mediate alternative patterns of RNA processing of cellular and viral transcripts (Caceres et al.; Yang et al.; Mayeda et al., EMBO J. 13: 5483-95 (1994); Caputi et al., EMBO J. 18: 4060-67 (1999); Nissim-Rafinia et al., Hum. Mol. Genet. 9: 1771-78 (2000); Bilodeau et al., J. Virol. 75: 8487-97 (2001)).

Catechins, including, but not limited to, EGCG (epigallocatechin gallate), ECG (epicatechin gallate) and GCG (gallocatechin gallate), are polyphenolic flavonoid compounds. They are found most abundantly in green tea, but also appear in black tea, grapes and chocolate, among others. Green tea catechins have demonstrated antioxidant activities, including scavenging of reactive species (such as superoxide, hydroxyl and peroxyl radicals), inhibition of lipid peroxidation and inhibition of the oxidation of low-density lipoproteins. Studies have also shown the catechins to have anticarcinogenic, anti-atherosclerotic, anti-inflammatory and antimicrobial activities. For example, EGCG has been reported to block carcinogenessis, inhibit the growth and induce apoptosis of cancer cells, modulate gene expression, and possess anti-microbial activity against bacteria, fungi, and viruses (Mukoyama et al., J. Med. Sci. Biol. 44: 181-86 (1991); Toda, et al., Microbiol. Immunol. 36: 999-1001 (1992); Okabe, et al., Jpn. J. Cancer Res. 88: 639-43 (1997); Yang, et al., Biofactors 13: 73-79 (2000); Abe, et al., Biochem. Biophys. Res. Commun. 281: 122-25 (2001); Okabe, et al., Biol. Pharm. Bull. 24: 883-86 (2001); Kazi, et al., In vivo 16: 397-403 (2002)).

In FD patients, the causative mutation results in the preferential use of an intron-distal 5′ splice site, the consequence of which is the exclusion of exon 20 and the generation of a truncated IKAP. The FD-causing mutation's position and leaky nature suggested that the mutation's impact might be moderated by altering the level of splice-regulating proteins. It has been reported that EGCG has the ability to down-regulate hnRNP A2/B1 protein (a trans-activating factor that encourages the use of intron-distal 5′ splice sites) and gene expression (Fujimoto et al., Int. J. Oncol. 20: 1233-39 (2002)).

The observed ability of tissues and cells derived from individuals with FD to produce some exon 20-containing, or wild-type, transcripts (Anderson et al., Biochem. Biophys. Res. Commun. 306: 303-09 (2003); Cuajungco et al., Am. J. Hum. Genet. 72: 749-58(2003)), suggested that the FD phenotype might be modulated through the production of variable amounts of the functional gene product. Anderson et al. (2003) demonstrated that tocotrienols, members of the vitamin E family, can up-regulate transcription of the IKBKAP gene. This increased expression results in an increased production of both the truncated and full-length transcripts and an increase in the amount of functional IKAP.

### SUMMARY OF THE INVENTION

It is an object of the present invention to evaluate the effect of catechins on mRNA splicing, such as mRNA splicing in a normal or diseased cell, particularly, the effect of epigallocatechin gallate (EGCG) and related catechins on IKBKAP transcription in cells, particularly, FD-derived cells. It is also an object of the present invention to identify whether EGCG and related catechin treatment of FD-derived cells can increase the level of exon 20-containing IKAP transcripts, i.e., the wild-type IKAP transcripts, and functional IKAP protein.

In one aspect, the present invention provides a method for modulating mRNA splicing in a cell by contacting the cell with an effective amount of at least one catechin, preferably, EGCG, ECG or GCG and combinations thereof. A particular aspect of the present invention is directed to elevating the level of the wild-type IKBKAP-encoded transcript and functional protein in a cell by contacting the cell with an effective amount of catechins, preferably, EGCG.

In another aspect, the present invention provides a method for treating an individual having FD by providing an effective amount of at least one catechin to the individual, preferably through an oral route.

In still another aspect, the present invention provides a method for treating an individual having FD by providing an effective amount of at least one catechin and one or more tocotrienols to the individual, preferably through an oral route.

In yet another aspect, an individual having FD is treated by providing an effective amount of at least one catechin and one or more tocotrienols in combination with one or more tocopherols.

In a further aspect, the present invention provides a kit for treating an individual having FD. The kit contains an effective amount of at least one catechin and, optionally, one or more tocotrienols and one or more tocotrienols in combination with one or more tocopherols, and instructions that typically provide suitable dosages and dosing schedules effective for treatment of FD. The kit can also include a pharmaceutically acceptable carrier.

### BRIEF DESCRIPTION OF THE DRAWINGS

FIG. 1 depicts an analysis of IKAP exon 20 sequence for ESS motifs. The 74 bp nucleotide sequence of IKAP was analyzed for putative ESS motifs. Two were found, each matching a reported ESS consensus sequence, PyTAG, and are presented in bold.

FIG. 2 depicts a real-time RT-PCR analysis of hnRNP A2/B1 transcripts in EGCG-treated FD-derived GM04663 cells. Cultures were treated for 24 h with varying concentrations of EGCG. The relative amounts of hnRNP A2/B1 RNA were determined by real-time RT-PCR and are presented as changes in the threshold cycle (ΔC_{T}) relative to RNA levels from untreated cells. Results presented represent mean values obtained in three experiments, each done in triplicate.

FIG. 3 depicts a real-time RT-PCR analysis of hnRNP A2/B1, hnRNP A1, and IKAP RNA levels in EGCG-treated cells. GM00850 (FD-derived), GM04663 (FD-derived), and GM02912 (normal) cells were treated for varying times with 50 μg/ml EGCG. The relative amounts of hnRNP A2/B1, hnRNP A1, wild-type IKAP (exon 20-containing), mutant IKAP (exon 20-lacking), and total IKAP (exon 34-35) RNA were determined by real-time RT-PCR and are presented as changes in the threshold cycle (ΔC_{T}) relative to RNA levels from untreated cells. The panels on the left show changes in hnRNP RNA levels and those on the right, changes in IKAP RNA levels. Results presented represent mean values obtained in three experiments, each done in triplicate.

FIG. 4 depicts an RT-PCR analysis of wild-type (exon 20-containing) and mutant (exon 20-lacking) IKAP transcripts in EGCG-treated cells. GM00850 (FD-derived), GM04663 (FD-derived), and GM02912 (normal) cells were treated for 24 h with 50 μg/ml EGCG. The relative amounts of wild-type (434 nt) and mutant (361 nt) IKAP transcripts were determined in RT-PCR reactions with primers spanning exons 19-23. The products were analyzed on 2% agarose gels. Results presented represent a typical result obtained.

FIG. 5 depicts Western blot analysis of hnRNP A2/B1, hnRNP A1, and IKAP levels in EGCG-treated cells. Two FD-derived cell lines, GM00850 and GM04663, were treated for 24 h with 50 μg/ml EGCG. Protein extracts were fractioned by SDS-PAGE, blotted onto nitrocellulose, and probed with antibodies against hnRNP A2/B1, hnRNP A1, and IKAP. Shown in (A) are results obtained in untreated (−) and EGCG-treated (+) cells. The blots were analyzed densitometrically to determine the % increase in the amounts of IKAP produced by EGCG treatment relative to untreated cells (B). Results presented represent a typical result obtained.

FIG. 6 depicts a real-time RT-PCR and Western blot analysis of IKAP levels in EGCG and tocotrienol treated cells. The FD-derived cell line, GM04663, was treated with 5 ug/ml EGCG, 6.25 ug/ml δ-tocotrienol or a combination of the two for 24 h. The relative amounts of exon 20-containing (wild-type) RNA were determined by real-time RT-PCR and are presented as changes in the threshold cycle (ΔC_{T}) relative to RNA levels from untreated cells (A). Results presented represent mean values obtained in three experiments, each done in triplicate. Protein extracts were subjected to Western blot analysis and probed with IKAP antibody (B). Results presented represent a typical result obtained. The blots were analyzed densitometrically to determine the % increase in the amounts of IKAP produced by the various treatments relative to untreated cells (C).

FIG. 7 demonstrates expression of IKAP RNA in post-mortem tissue samples. RT-PCR, using primers located in exon 20 and spanning exons 21 and 22, was performed on RNA isolated from post-mortem tissue samples from two individuals with FD. The resulting amplified products were fractionated on a 2% agarose gel.

FIG. 8 depicts a real-time RT-PCR analysis of IKAP RNA in cells treated with different catechins.

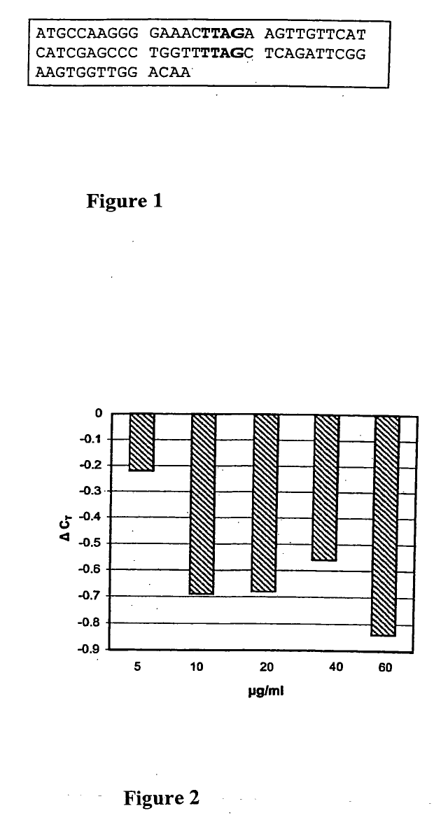

### DETAILED DESCRIPTION OF THE INVENTION

Familial Dysautonomia (FD) is a neurodegenerative genetic disorder caused by mutations in the IKBKAP gene that encodes the IKB kinase complex-associated protein (IKAP). The major mutation causes aberrant splicing, resulting in the production of a truncated form of IKAP. Tissues from individuals homozygous for the major mutation contain both mutant and wild-type IKAP transcripts.

The present invention discovered that catechins, which are polyphenolic flavonoid compounds, can modulate mRNA splicing, such as mRNA splicing in a normal cell or diseased cell. Catechins useful in accordance with the present invention include, but are not limited to, epigallocatechin gallate (EGCG) and related catechins, such as ECG and GCG. By “EGCG-related catechins” is meant catechins that have similar or the same function, structure or effects as EGCG.

In particular, the present invention provides that catechins can increase the amount of the exon 20-containing IKAP transcript and functional protein in catechin, e.g., EGCG, treated cells, particularly, FD-derived cells. The present invention also discovered that combined treatment of cells with catechins and tocotrienol, which up-regulates IKBKAP transcription, results in a synergistic production of the exon 20-containing IKAP mRNA and full-length IKAP protein. Thus, EGCG-related catechins can increase the amount of the wild-type IKBKAP-encoded transcript and functional protein. The present invention demonstrates that catechins and EGCG-related catechins provide therapeutic modalities for individuals with FD.

The present invention also contemplates that catechins can modulate mRNA splicing in normal or diseased cells, such as cancer cells. A particular embodiment of the present invention encompasses modulating mRNA splicing in cells. For example, in one embodiment, the present invention contemplates increasing wild-type mRNA transcripts in a breast cancer cell, e.g., a cell having one or more BRCA mutations.

In one embodiment, the present invention provides a method for modulating mRNA splicing, preferably, increasing wild-type transcripts, more preferably, increasing the amount of the wild-type IKBKAP-encoded transcript, in a cell, particularly a FD-derived cell, by contacting the cell with an effective amount of at least one catechin, preferably an EGCG-related catechin, more preferably, EGCG.

Particularly, the present invention provides that EGCG and EGCG-related catechins can increase the amount of the exon 20-containing IKAP transcript in EGCG or EGCG-related catechin treated FD-derived cells. Without intending to be bound by any specific mechanism, it is believed that such effect results from reducing the level of hnRNP A2/B1 in the cells. It is believed that supplementation of catechins, such as EGCG, can down-regulate the expression of hnRNP A2/B1, which is a trans-activating factor that encourages the use of intron-distal 5′ splice sites. It is believed that reduced levels of hnRNP A2/B1 results in an increased level of correctly spliced IKAP transcript and normal IKAP protein in FD-derived cells or in an individual having FD. It is further believed that such results can impact in the treatment of FD.

By “cell” is meant to include normal or diseased cells derived from an individual or cells within an individual. The individual can be an individual with FD, cancer and/or other disease condition, or a normal individual. By “FD-derived cell” is meant a cell isolated or derived from an individual with FD or a cell within an individual with FD.

By “contacting the cell with an effective amount of catechin” is meant to include contacting the cell in vitro with the catechin, as well as providing or administering the catechin, e.g., EGCG to an individual such that the cell is exposed to the catechin in vivo.

The term “IKBKAP” has been used herein to refer to the gene, whereas “IKAP” has been used to refer to the mRNA transcript or the encoded protein.

The level of alternatively spliced transcript, e.g., wild-type IKBKAP-encoded transcript (i.e., exon-20 containing IKAP transcript), can be determined by using a variety of methods well known to those skilled in the art, including, inter alia, Northern Blot analysis, RT-PCR. An elevated level of a particular mRNA splice variant in a cell, e.g., IKBKAP-encoded transcript in an FD-derived cell treated with EGCG, can be observed when compared with untreated cells.

The elevated level of the wild-type IKBKAP-encoded transcript can result in an elevated level of the IKAP protein. Accordingly, in another embodiment, the present invention provides a method of elevating the level of the IKAP protein in a cell, particularly an FD-derived cell, by contacting the cell with an effective amount of at least one catechin, such as EGCG or EGCG-related catechins.

The level of the IKAP protein produced in a cell can be determined by using a variety of methods well known to those skilled in the art, including Western Blot analysis. An elevated level of the IKAP protein in FD-derived cells treated with EGCG is observed when compared with untreated cells.

EGCG-related catechins can be readily obtained from various commercial sources, such as Calbiochem (San Diego, Calif.).

The amount of catechins that is effective to potentiate a catechin-induced elevation of mRNA splice variants and protein variants thereof, e.g., IKBKAP transcript and functional IKAP, in a cell, particularly a FD-derived cell, may vary, depending on the manner by which the cell is brought into contact with the catechins. In general, a suitable dose of catechin in the range of about 15 mg to about 120 mg per day is effective to elevate the level of the wild-type IKBKAP-encoded transcript and functional protein in cells in vivo within an individual. Preferably, an amount in the range of about 30 mg to about 60 mg per day is provided to an individual. When cells are exposed to catechins in vitro, an amount of catechins in the range of about 12.5 to about 50 μg/ml, preferably, about 50 μg/ml, is effective.

In another embodiment, the present invention provides a method for treating an individual having FD by providing an effective amount of at least one catechin, preferably, EGCG, ECG or GCG, more preferably, EGCG to the individual, preferably, through an oral route.

The term “treating” is meant to ameliorate, inhibit or eliminate the symptoms associated with FD, or to improve the health of an individual having FD. Symptoms known to be associated with FD as a result of the malfunctioning of the autonomic system (such as the blood pressure control system) include hypertensive crisis, lack of sensitivity to pain, retching, and a lack of overflow tears, among others.

In accordance with the present invention, catechins, such as EGCG, can be provided to an individual having FD as young as a few days old.

Catechins can be provided to an FD individual throughout their lifetime, from birth, or even prenatally via maternal ingestion. The amount of catechins that is effective may depend on the age, condition and body weight of a particular individual having FD. In general, a suitable dose of catechins in the range of about 15 to about 120 mg per day is effective. Preferably, an amount in the range of about 30 to about 60 mg per day is provided to an FD patient. EGCG-related catechins can be provided to an individual having FD for as long as necessary to treat FD. The duration of the treatment can be determined by the skilled artisan by routine experimentation.

The present invention also provides that combined treatment of cells with catechins, such as EGCG, ECG or GCG, and one or more tocotrienols results in a synergistic production of correctly spliced transcript and full-length protein in cells.

In a copending application (U.S. Application No. 60/571,367, filed on May 14, 2004 (“the '367 application”), the invention provides that tocotrienols, members of the vitamin E family, have the ability to increase transcription of IKAP mRNA in cells, including normal or FD-derived cells, with corresponding increases in the correctly spliced IKAP transcript and normal IKAP protein.

According to the '367 application, a tocopherol can enhance a tocotrienol-induced elevation of IKAP mRNA levels, even though the tocopherol alone does not have an impact on IKAP mRNA levels. Tocotrienols suitable for use in the present invention include α, β, γ, and δ tocotrienols. Tocophenols suitable for use in the present invention include α, β, γ, and δ tocopherols. The '367 application is incorporated herein by reference.

In one embodiment, the present invention provides a method for elevating the level of the wild-type IKBKAP transcripts or the functional IKAP protein in a cell by contacting the cell with an effective amount of at least one catechin, such as EGCG, ECG or GCG, and one or more tocotrienols.

In another embodiment, the present invention provides a method for treating an individual having FD by providing an effective amount of at least one catechin, such as EGCG, ECG or GCG, and one or more tocotrienols to the individual, preferably through an oral route.

In still another embodiment, an individual having FD is treated by providing an effective amount of at least one catechin, such as EGCG, ECG or GCG, and one or more tocotrienols in combination with one or more tocopherols.

The term “in combination” does not require simultaneous administration of tocopherols and tocotrienols, so long as both tocopherols and tocotrienols are given to the cell or the individual.

Catechins, such as EGCG, GCG or ECG, alone or in combination with tocotrienols can be provided in a pharmaceutical carrier for use in the present methods. As used herein, a pharmaceutically acceptable carrier includes any and all solvents, dispersion media, isotonic agents and the like. Except insofar as any conventional media, agent, diluent or carrier is detrimental to the recipient or to the therapeutic effectiveness of the active ingredients contained therein, its use in practicing the methods of the present invention is appropriate. The carrier can be liquid, semi-solid, e.g., pastes, or solid carriers. Examples of carriers include water, saline solutions, alcohol, oils, sugar, gel, lipids, liposomes, resins, porous matrices, binders, fillers, coatings, preservatives and the like, or combinations thereof. Carriers that are capable of controlled release, e.g., a controlled release matrix, are also contemplated by the present invention.

In accordance with the present invention, catechins and tocotrienols can be combined with a pharmaceutical carrier in any convenient and practical manner, e.g., by admixture, solution, suspension, emulsification, encapsulation, absorption and the like, and can be made in formulations such as tablets, capsules, powder, syrup, suspensions that are suitable for injections, implantations, inhalations, ingestions or the like.

According to the present invention, catechins and tocotrienols can be provided to an individual having FD by standard routes, including the oral, enteral and parenteral routes. Preferably, an individual having FD is given catechins or catechins and tocotrienols orally.

In a further embodiment, the present invention provides a kit for treating an individual having FD, which contains an effective amount of at least one catechin and instructions that typically set forth suitable dosages and dosing schedules effective for treatment of FD. The kit can also include a pharmaceutically acceptable carrier that is described hereinabove.

In a further embodiment, the present invention provides a kit for treating an individual having FD. The kit contains an effective amount of at least one catechin and one or more tocotrienols. In a even further embodiment, the kit contains an effective amount of at least one catechin and one or more tocotrienols in combination with one or more tocopherols, and instructions that typically set forth suitable dosages and dosing schedules effective for treatment of FD. The kit can also include a pharmaceutically acceptable carrier.

The present invention is further illustrated by the following non-limiting examples.

### EXAMPLE 1

### Detection of the Wild Type IKAP Transcript in FD-Derived Tissues

RNA was isolated from a variety of tissues of FD-affected individuals to determine the presence of wild-type IKAP transcript. Frozen tissue was homogenized with a Tissue Tearor (Biospec Products) in Lysis/Binding Solution (Ambion) and the RNA was purified using the RNAqueous Total RNA Isolation Kit (Ambion). The RNA was amplified using a primer recognizing the sequence encoded in exon 20 of the IKBKAP gene and a primer that spans exons 21 and 22 by following the instruction of the GeneAmp EZ rTth RNA PCR Kit (Applied Biosystems). 25 ng of RNA were used as template in a reaction volume of 20 μl. One-step RT-PCR was carried out as follows: one cycle of 58° C. for 45 min and 94° C. for 2 min, followed by 45 cycles of 94° C. for 30 sec, 58° C. for 30 sec, and 72° C. for 30 sec, and then a final extension of 72° C. for 7 min. PCR products were then analyzed on a 2% agarose gel.

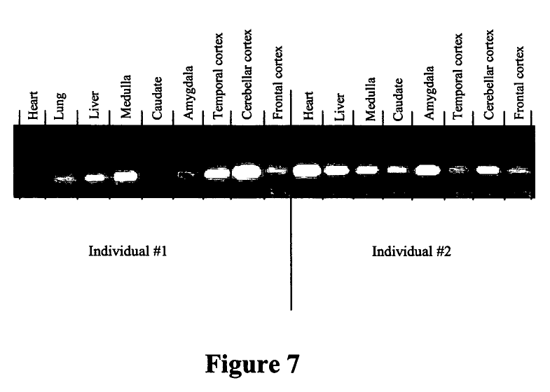

As shown in FIG. 7, exon 20-containing IKAP transcript was detected in all of the FD-derived tissues studied. This observation is consistent with a report by Cuajungco et al. See Cuajungco et al., Am. J. Hum. Genet. 72: 749-58(2003).

### EXAMPLE 2

### EGCG Decreased the Level of hnRNP A2/B1 Transcripts Present in FD-Derived Fibroblast Cells

The GM00850 and GM04663 cell lines, homozygous for the IVS20^{+6T→C} FD-causing mutation, and the GM02912 cell line, derived from an unaffected individual, were obtained from the NIGMS Human Genetic Mutant Cell Repository. LA1-55n cells were provided by Dr. Robert A. Ross. EGCG and δ-tocotrienol were purchased from Calbiochem. A monoclonal antibody generated against a peptide encoded by exons 23-28 of IKBKAP was purchased from BD Biosciences. The 4B10 and DP3B3 monoclonal antibodies, which recognize hnRNP A1 and hnRNP A2/B1, respectively, were provided by Dr. Gideon Dreyfuss.

In order to identify agents that could modulate the level of the wild type IKAP transcript produced in FD-derived cells, either by increasing transcription or modulating splicing, FD-derived fibroblast cells (GM04663) were treated with EGCG. RNA was isolated from GM04663 cells treated for 24 h with varying concentrations of EGCG was subjected to real-time RT-PCR analysis using a pair of primers that recognize both of the hnRNP A2/B1 transcripts. More specifically, FD-derived fibroblast cells, seeded in 96-well plates were treated in triplicate for approximately 24 h unless noted otherwise. After treatment, the cells were washed once with PBS and then lysed in 50 μl Cell-to-cDNA lysis buffer (Ambion) at 75° C. for 12 min. After one freeze-thaw cycle, 4 μl of lysate was used in 20 μl RT-PCR. The Quantitect SYBR Green RT-PCR Kit (Qiagen) was used for real-time RT-PR analysis of the relative quantities of the exon 20-containing transcript (wild-type), the exon 20-lacking transcript (mutant), and exon 34-35 containing transcript that is unaffected by the FD-causing mutation (total), as well as the hnRNP A2/B1 and hnRNP A1 transcripts.

The primers used for these analyses were as follows: for the exon 20-containing transcript, 5′-AGTTGTTCATCATCGAGC-3′ (SEQ ID NO: 3) and 5′-CATTTCCAAGAAACACCTTAGGG-3′ (SEQ ID NO: 4); for exon 20-lacking transcript, 5′-CAGGACACAAAGCTTGTATTACAGACTT-3′ (SEQ ID NO: 5) and 5′-CATTTCCAAGAAACACCTTAGGG-3′ (SEQ ID NO: 6); for exon 35-35 containing transcript, 5′-GAGATCATCCAAGAATCGC-3′ (SEQ ID NO: 7) and 5′-GGTAGCTGAATTCTGCTG-3′ (SEQ ID NO: 8); for hnRNP A2/B1 transcript, 5′-GAGTTGTTTCTCGAGCAG-3′ (SEQ ID NO: 9) and 5′-TGATCTTTTGCTTGCAGG-3′ (SEQ ID NO: 10); and for hnRNP A1 transcript, 5′-TCGTGGAGGAAACRRCAGRG-3′ (SEQ ID NO: 11) and 5′-TGTAGCTTCCACCACCTCCA-3′ (SEQ ID NO: 12). Primers were used at a concentration of 0.5 μM. An ABI PRISM 7000 Sequence Detection System (Applied Biosystems), programmed as follows, was used to perform the real-time RT-PCR and analysis: 50° C.×30 min and 95° C.×15 min for one cycle, followed by 40 cycles of 94° C.×15 s, 57-60° C.×30 s, and 72° C.×30 s. To present relative amounts of PCR product obtained, results are expressed as changes in the threshold cycle (ΔC_{T}) compared to untreated cells. The threshold cycle refers to the PCR cycle at which the fluorescence of the PCR is increased to a calculated level above background. A change of 1.0 in C_{T}, assuming 100% PCR efficiency, would reflect a twofold change in the starting amount of the RNA template that was amplified.

To control for the amount of RNA present in the samples, RT-PCR amplification of ribosomal 18S RNA was performed on all cell lysates. The use of the ribosomal 18S RNA has been shown to be an effective control for the quantity of RNA present in samples (D. Goidin et al., Anal. Biochem. 295: 17-21 (2001)). For this analysis, TaqMan Ribosomal RNA Control Reagents were used with the TaqMan EZ RT-PCR Kit (Applied Biosystems) as per the manufacturer's protocol.

A clear concentration-dependent decrease in the level of the hnRNP A2/B1 transcript was observed (FIG. 2). It was observed that primers that recognized the A2 and B1 transcripts independently gave identical results.

### EXAMPLE 3

### EGCG Increased the Level of the Exon 20-Containing IKAP mRNA

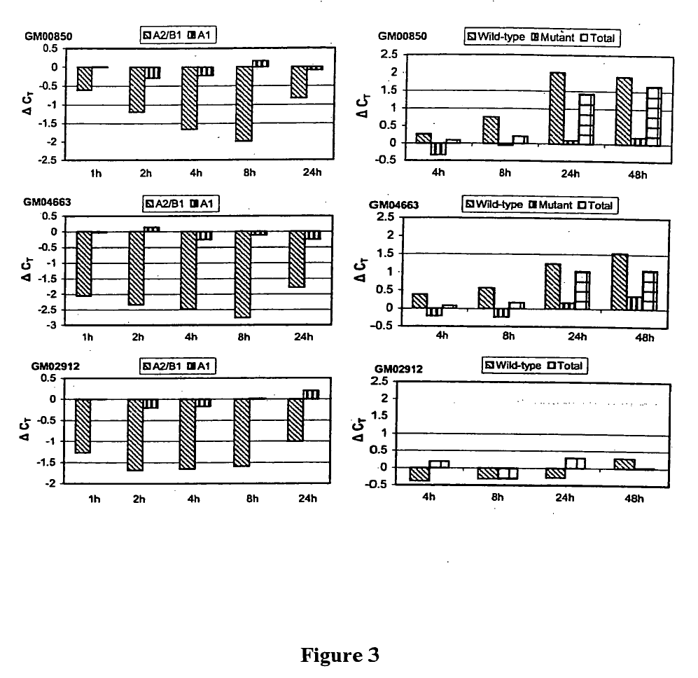

To determine the effect of EGCG on the cellular level of the exon 20-containing (wild type) IKAP mRNA, FD-derived and normal fibroblasts were treated with 50 μg/ml EGCG for varying lengths of time. The levels of hnRNP A2/B1, hnRNP A1, IKAP exon 20-containing (wild type), IKAP exon 20-lacking (mutant), and IKAP exon 34-35-containing (total) transcripts were determined (FIG. 3).

It was observed that EGCG treatment results in a time-dependent reduction in the level of the hnRNP A2/B1 transcript while having no effect on the level of the hnRNP A1 transcript. The absence of an impact on hnRNP A1 levels revealed that EGCG does not have a generalized effect on all members of the hnRNP A/B family. It was further observed that exon 20-containing IKAP transcript was elevated in FD-derived cells but not in normal cells. The elevated presence of this transcript appears to be due to a modulation of the splicing process and not an increase in transcription, as the level of the exon 20-lacking transcript was not affected by EGCG and normal cells failed to exhibit an increase in the level of the exon 20-containing transcript.

The effect of EGCG treatment was further monitored by RT-PCR analysis using primers located in exons 19 and 23 capable of amplifying both the normal and mutant forms of the IKAP transcript. 4 μl of cell lysates produced as described above was used in 20 μl RT-PCR using the One Step RT-PCR Kit (Qiagen) with the following primers which recognize sequences in exons 19 and 23 of IKBKAP: 5′-GCAGCAATCATGTGTCCCA-3′ (SEQ ID NO: 1) and 5′-TAGCATCGCAGACAAGGTC-3′ (SEQ ID NO: 2). The RT-PCR was carried out as follows: one cycle of 50° C.×30 min and 95° C.×15 min, followed by 41 cycles of 94° C.×20 s, 60° C.×30 s and 72° C.×15 s, and then a final extension of 72° C.×2 min. PCR products were analyzed on a 2% agarose gel.

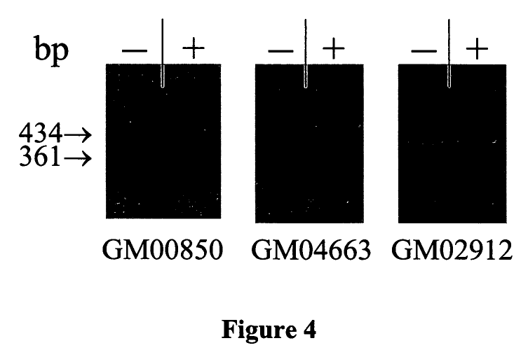

A clear enhanced presence of exon 20-containing transcript is present in the EGCG treated FD-derived cells while having no effect on the normal cells (FIG. 4).

### EXAMPLE 4

### EGCG Suppressed the Level of hnRNP A2/B1

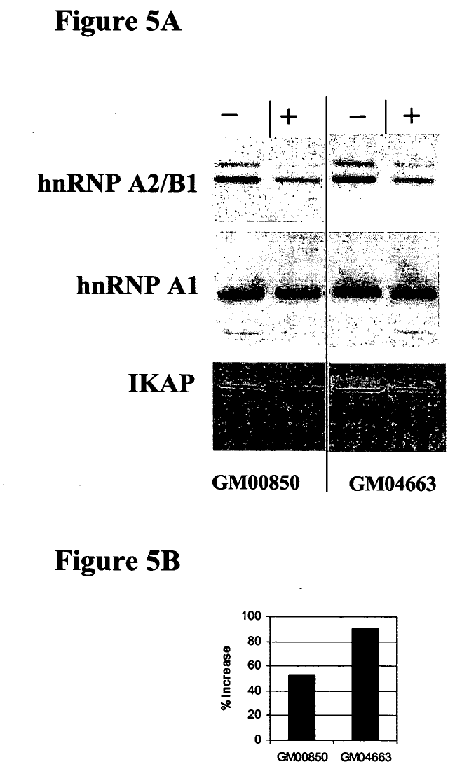

Western blot analysis using antibodies recognizing hnRNP A1 and hnRNP A2/B1 confirms the ability of EGCG treatment to suppress the level of hnRNP A2/B1 while hnRNP A1 levels are unaffected (FIG. 5). Antibody to IKAP that recognize the full-length protein was used to probe Western blots on which cellular extracts from EGCG-treated FD-derived GM00850 and GM04663 fibroblast cells were fractionated. A clear increase in the amount of IKAP present in FD-derived fibroblasts was detected (FIG. 5).

GM00850 and GM04663 cells treated for 24 h with 50 μg/ml of EGCG were washed twice with PBS and lysed in 0.5 M Tris-HCl, pH 6.8, containing 1.4% SDS. Western blot analysis was performed essentially as described by Rubin et al. (Proc. Natl. Acad. Sci. USA 82: 6637-41 (1985)). Equal amounts of protein fractionated on a 7% NuPAGE Tris-Acetate Gel (Invitrogen) were blotted onto nitrocellulose (Bio-Rad) and probed overnight with a monoclonal antibody (BD Biosciences) to IKAP that is directed against the carboxyl end (exons 23-28) of IKAP and therefore recognizes the full-length protein. The blot was then washed and probed with a goat anti-mouse antibody conjugated to alkaline phosphatase (Promega), followed by detection with Western Blue Substrate solution (Promega). All blots were also probed with an anti-actin antibody (Oncogene) to confirm equal protein loading.

### EXAMPLE 5

### Simultaneous Tocotrienol and EGCG Supplementation in FD-Derived Cell Lines Elevated the Level of Functional IKAP mRNA

Noting the recently observed ability of tocotrienols to elevate transcription of IKBKAP, an evaluation of the response of FD-derived cells to the combined treatment of tocotrienols and EGCG was conducted.

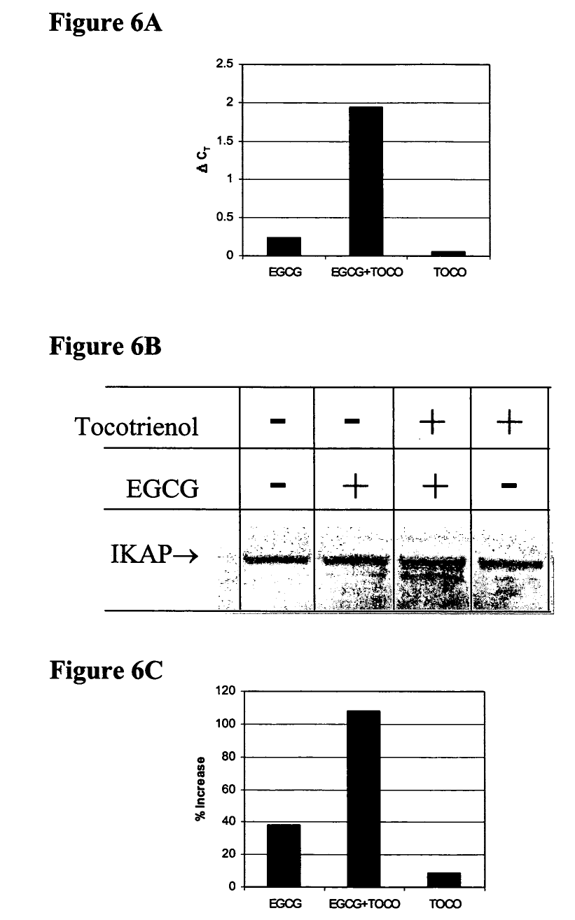

GM04663 cells were treated with a combination of 8-tocotrienol and EGCG, at doses that by themselves did not result in a significant elevation in the level of the wild-type of IKAP transcript, and the levels of the exon 20-containing IKAP transcript and full length IKAP protein were determined. A clear synergistic increase in the level of the transcript and protein was observed (FIG. 6). Similar results were obtained with the GM00850, FD-derived cell line.

### EXAMPLE 6

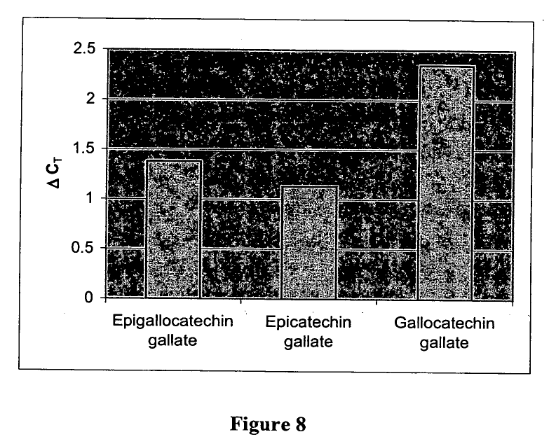

A real-time RT-PCR analysis of IKAP RNA in cells treated with different catechins was conducted. GM00850 cells were treated with either no catechins or with 50 μg/ml of either epigallocatechin gallate (EGCG), epicatechin gallate (ECG) or gallocatechin gallate (GCG) for 48 hours. The relative amount of wild-type (exon 20-containing) IKAP RNA was determined by real-time RT-PCR and is presented as changes in threshold cycle (ΔC_{T}) relative to RNA levels from untreated cells. The result is illustrated by FIG. 8.

What is claimed is:

1. A method for modulating the level of an alternatively spliced mRNA transcript in a cell comprising contacting the cell with an effective amount of at least one catechin.

2. The method of claim 1, wherein said catechin is selected from a group consisting of epigallocatechin gallate, epicatechin gallate and gallocatechin gallate.

3. The method of claim 1, wherein said mRNA transcript is IKBKAP transcript.

4. A method of elevating the level of the IKAP protein in a cell comprising contacting the cell with an effective amount of at least one EGCG-related catechin.

5. The method of claim 3 or 4, wherein said cell is derived from an individual or within an individual.

6. The method of claim 5, wherein said individual has Familial Dysautonomia.

7. The method of claim 3 or 4, wherein said cell is brought into contact in culture with said catechin at a concentration in the range of about 12.5 μg/ml to about 50 μg/ml.

8. The method of any one of claims 1 to 4, further comprising contacting the cell with an effective amount of one or more tocotrienols.

9. The method of claim 8, further comprising contacting the cell with an effective amount of one or more tocopherols.

10. A method of elevating the level of the wild-type IKBKAP transcript in an individual comprising providing an effective amount of at least one catechin to said individual.

11. A method of elevating the level of the IKAP protein in an individual comprising providing an effective amount of at least one catechin to said individual.

12. A method for treating an FD individual comprising providing an effective amount of at least one catechin to said individual.

13. The method of any one of claims 10-12, wherein said catechin is provided to said individual in an amount of about 15 mg to about 120 mg per day.

14. The method of claim 13, wherein said catechin is provided to said individual orally, enterally or parenterally.

15. The method of any one of claims 10-12, further comprising providing an effective amount of one or more tocotrienols to said individual.

16. The method of any one of claims 15, further comprising providing an effective amount of one or more tocopherols to said individual.

17. The method of claim 16, wherein said tocotrienols are selected from the group consisting of α, β, γ, or δ tocotrienols, or a combination thereof.

18. A kit for treating an individual having FD, comprising an effective amount of at least one catechin and instructions for use.

19. The kit of claim 18, further comprising an effective amount of one or more tocotrienols and tocopherols.
